(19) United States
(12) Patent Application Publication
(10) Pub. No.: US 2004/0116394 A1
(43) Pub. Date: Jun. 17, 2004

(54) Method for treating cancer using betulinic acid rich herbal extract

(75) Inventors: Rama Mukherjee, DLF City (IN); Dhiraj Khattar, Ghaziabad (IN); Manu Jaggi, DLF City (IN); Anu T. Singh, Ghaziabad (IN); Mukesh Kumar, Delhi (IN); Honey Bala, New Delhi (IN)

Correspondence Address: LADAS & PARRY, 26 WEST 61ST STREET, NEW YORK, NY, 10023, US

(73) Assignee: DABUR RESEARCH FOUNDATION

(21) Appl. No.: 10/319,374

(22) Filed: Dec. 13, 2002

(51) Int. Cl. A61K 31/56; A61K 35/78

(52) U.S. Cl. 514/169; 424/756

(57) ABSTRACT

This invention relates to an orally effective herbal extract-based composition having broad-spectrum anticancer activity, more specifically a method of treating, inhibiting and/or preventing malignant tumors of the colon, intestine, stomach, breast, melanoma, glioblastoma, lung, cervix, ovary, prostate, oral cavity, larynx, liver, pancreas, kidney, bladder, endothelial cells, leukemia and myeloma using a herbal extract of Zizyphus, rich in betulinic acid. An advantage of the extract is that the betulinic acid has low systemic toxicity. The extract inhibits Protein Kinase C activity of cancer cells and induces apoptosis.

### FIELD OF THE INVENTION

This invention relates to an orally effective herbal extract-based composition having broad-spectrum anticancer activity, more specifically a method of treating, inhibiting and/or preventing malignant tumors of the colon, intestine, stomach, breast, melanoma, glioblastoma, lung, cervix, ovary, prostate, oral cavity, larynx, liver, pancreas, kidney, bladder, endothelial cells, leukemia and myeloma using a herbal extract of Zizyphus, rich in betulinic acid as well as having low systemic toxicity. The extract inhibits Protein Kinase C activity of cancer cells and induces apoptosis.

### BACKGROUND OF THE INVENTION

Any individual is at risk of developing cancer and the risk increases with aging over a lifetime. By an estimate from World Health Organization (WHO) about 10 million new cancer cases are occurring around the world annually and this number is expected to reach 15 million by the year 2015, with two thirds of these cases occurring in developing countries.

It may be noted that plants are considered a valuable resource for the discovery and development of novel, naturally derived agents to treat cancer. A few of the well known plant derived anticancer agents that have received U.S. FDA approval include paclitaxel, vinblastin, vincristine, topotecan, etoposide, teniposide, camptothecin, irinotecan etc.

The U.S. FDA approved its first single herb drug for Phase II Clinical Trials in 2001. An anti-cancer drug, called Kanglaite Injection, made by China's Zhejiang Kanglaite Pharmaceutical, is the first herbal drug approved by the FDA for clinical trials on humans. The US company Oncoherb has been designated to conduct the Phase II clinical trials. Kanglaite is developed from the liquid distilled from the seeds of the herb called Job's tears (Yiyiren). It is able to kill cancer cells by enhancing the immune system of the human body.

Certain plants in tropical countries have been known for centuries to have some curative values. One of these is the Zizyphus jujuba, which is used to treat a variety of common illnesses such as gastrointestinal disorders, insomnia, ulcers, and gingivitis. The genus Zizyphus (ber,jujube) belongs to the buckthorn family (Rhamnaceae). It is a genus of about 100 species of deciduous or evergreen trees and shrubs distributed in the tropical and subtropical regions of the world. Some species, like Z. jujuba and Z. mauritiana, occur in nearly every continent, whereas other species are restricted in their distribution to distinct areas. The fleshy drupes of several species are rich in sugars and vitamins, and this fact has made Zizyphus species important fruit trees for many centuries. In both China and India, Zizyphus trees have a long tradition of selection and cultivation, with the result that the species occurring in these countries (Z. mauritiana, Z. jujuba) are better known and more widely researched than those in other regions. The bark and the leaves in decoction are used as an astringent and for the treatment of dysentery, diarrhea and bowel irregularities. Its powdered form is used for dressing of old wounds and ulcers. In Cambodia, the bark is also prescribed for dysentery and gingivitis. The leaves are an ingredient used by some Benue tribe in prescription for gonorrhea. The Leaves in plaster form are used in strangury. A paste made from the tender leaves and twigs is applied to boils, abscesses, and carbuncles to promote suppuration. Thefruit is said to be nourishing, mucilaginous, pectoral and styptic, and is said to purify the blood and assist in digestion. The fruit is considered cooling, anodyne and tonic. The fruit of the wild variety is very acidic and astringent, the cultivated fruit is less acid. The fruit in China is employed to relieve coughs.

Bark infusions of Zizyphus have been employed in Northeastern Brazil as a remedy for fever (Soares et al, Braz J Med Biol Res, 20: 5, 1987, 599-601). The aqueous extract of the plant was shown to have antipyretic activity in rabbits rendered febrile by intravenous injection of E. coli endotoxin. Fever responses were significantly decreased by the oral administration of a bark infusion of Zizyphus. These results lend support to the popular use of infusions of this plant in folk medicine as a remedy for fever. In another study, the aqueous extract of Zizyphus showed hypoglycemic activity in alloxan induced diabetic rats (Adithan C. et al. Indian Journal of Pharmacology. 2000 32: S67-S80).

Laboratory animal studies of Zizyphus extract confirm a sedative effect, though the constituents that contribute this effect have not all been specifically identified (Tang Wand Eisenbrand G, Chinese Drugs of Plant Origin, 1992 Springer-Verlag, Berlin.). The only components of Zizyphus that are present in quantities likely to be responsible for the observed clinical effects are triterpenes. The unique triterpenes in this herb are known as jujubosides. Additionally, there are related triterpene compounds (such as betulinic acid and oleanolic acid) that are found in several other herbs.

Under the auspices of a National Cooperative Natural Product Drug Discovery Group supported by the National Cancer Institute, the potential antitumor activity of approximately 2500 extracts derived from plants collected from all over the globe was evaluated in a panel of enzyme based assays and in a panel of cultured human tumor cell lines. One such extract, prepared from the stem bark of Ziziphus mauritiana Lam. (Rhamnaceae), displayed selective cytotoxicity against cultured human melanoma cells (Nature Medicine, Pisha et al., Vol. 1, No. 10, pages 1046-1051, October 1995; WO 96/29068). As a result of bioactivity guided fractionation, betulinic acid, a pentacyclic triterpene, was identified as a melanoma-specific cytotoxic agent.

Betulinic acid can be derived from several natural (botanical) sources. It can also be chemically derived from betulin, a substance found in abundance in the outer bark of white birch trees (Betula alba). Betulinic acid has been found to selectively kill human melanoma cells (Nature Medicine, Vol.1(10),1995, WO 96/29068).

We have previously reported the anticancer activity of betulinic acid and its derivatives in cancers of prostate, lung, ovary, leukemias and lymphomas. (U.S. Pat. Nos. 6,048,847 and 6,214,814). These patents describe compounds and compositions for treating, inhibiting and/or preventing tumor growth and particularly, for treating, inhibiting and/or preventing the growth of leukemia, lymphomas, prostate, lung and ovarian cancers using a natural product-derived compound and its derivatives.

Further, antiangiogenic activity of betulinic acid and its derivatives was also recently reported by the applicants in U.S. Pat. Nos. 6,228,850 and 6,403,816 wherein betulinic acid and its derivatives were shown to inhibit the formation of tube-like-structures (TLS) of endothelial cells when grown on Matrigel coated surface. The endothelial cell anti-proliferative activity along with anti-TLS activity was shown to suggest the anti-angiogenic activity of betulinic acid derivatives.

The promising broad spectrum anticancer activity of betulinic acid prompted us and many other scientists to develop processes for isolation/extraction of betulinic acid and/or its precursor betulin.

Some of the common methods employed in the art for the extraction of betulinic acid have been summarized by the applicants (U.S. Pat. No. 6,264,998).

However, all these processes suffer from several major drawbacks. For example, the use of a boiling organic solvent, at standard pressure, in the extraction may destroy the useful compounds present in the bark.

Another drawback with the current extraction processes is that the organic solvents employed are hazardous, difficult to handle or difficult to dispose of. The typical organic solvents, which include methylene chloride, benzene, toluene and chloroform, are hazardous to humans (i.e., some are toxic or carcinogenic) and equally importantly—are hazardous to the environment. Considering the industrial scale on which the extraction processes would need to be performed in order to provide industrial quantities of betulin or betulinic acid, large quantities of organic solvents would be required. The high cost of disposing the organic solvents is an additional disadvantage of the current extraction procedures.

A need therefore exists for a method that can be used to extract betulin or betulinic acid without damaging or losing other useful compounds and without being harmful to the operator and the environment.

Further, betulinic acid is a highly hydrophobic molecule and is very difficult to solubilize and formulate, an observation evident from the fact that even after so many years of research on this molecule, solubilization of betulinic acid is still an unresolved issue. This is one of the main reasons for such slow progress of pre-clinical work on this molecule. It is also a known fact that such insoluble compounds exist in better solubilised form in nature in the natural resources.

This prompted the applicants to devise a process/method for the preparation of a herbal extract rich in betulinic acid from the bark of Ziziphus jujuba, which could be used for the treatment of cancer in humans. Alternatively, the described method of preparation of herbal extract can also use other Zizyphus varieties, including, but not limited to Z. mauritiana, Z. rotundifolia, Z. mucronata, Z. nummularia, Z. lotus, Z. spina-christi, Z. obtusifolia.

We report here for the first time anticancer activity of herbal extract rich in betulinic acid against cancers of colon, intestine, stomach, breast, melanoma, glioblastoma, lung, cervix, ovary, prostate, oral cavity, larynx, liver, pancreas, kidney, bladder, endothelial cells, leukemia and myeloma both in vitro and in vivo. Human cancer cell lines have been extensively used in cancer research. Further, these cell lines represent a standard practice and norm for testing molecules for anticancer activity in vitro, and for prediction of their efficacy in xenografts in vivo. (Br J Cancer. May 18, 2001; 84(10):1289-90, “Semin Oncol December 1992; 19(6):622-38). Additionally the herbal extract extends the spectrum of reported anticancer activity of Betulinic acid. Betulinic acid has anticancer activity in leukemias, lymphomas, prostate, lung, ovarian cancer (U.S. Pat. No. 6,048,847) and in cancers of the colon, Breast, Glioblastoma, cervical & the oral cavity, (U.S. Pat. No. 5,962,527) The observed anticancer activities in the different cancers and the underlying mechanisms determining them suggest the utility of the extract for treatment/inhibition and prevention of tumour development in these cancers.

Accordingly, one aspect of this invention is to prepare a herbal extract rich in betulinic acid and compositions thereof for treating, inhibiting and/or preventing cancer more specifically the cancers of the colon, intestine, stomach, breast, melanoma, glioblastoma, lung, cervix, ovary, prostate, oral cavity, larynx, liver, pancreas, kidney, bladder, endothelial cells, leukemia and myeloma.

Another aspect of the present invention is to provide a simple and cost effective method for the preparation of a betulinic acid rich herbal extract having broad-spectrum anticancer activity.

Another aspect is to provide a process, which does not involve tedious step of chromatographic technique at any stage for the preparation of herbal extract rich in betulinic acid.

Still another aspect is to provide an eco-friendly process, which provides complete and efficient extraction of the useful compounds including betulinic acid from the replenishable source of raw material used.

Yet another aspect is to provide a process wherein the solvent used is non-toxic to humans and at the same time can be recycled in various extractions.

Another aspect of the invention is to prepare a herbal extract rich in betulinic acid for oral administration. Another aspect is that the herbal extract has low systemic toxicity.

### SUMMARY OF THE INVENTION

The invention relates to a herbal extract rich in betulinic acid having broad-spectrum anticancer effect, primarily mediated by the inhibition of Protein Kinase C activity and induction of the apoptotosis of cancer cells. The extract obviates the need for solubilizing the difficult to solubilize active principle (e.g. betulinic acid) that use conventional solvents, which may be hazardous for systemic administration. The present invention also relates to the preparation of a herbal extract having anticancer activity. The invention provides methods for isolating the chemical constituents of Zizyphus bark. Specifically, the present invention provides a method that can be used to prepare an extract rich in betulinic acid from Zizyphus bark without damaging other compounds remaining in the Zizyphus bark. In addition, the extraction process employs solvents that are safe (non-toxic and non-carcinogenic), easy to handle, environmentally-friendly, inexpensive, and recyclable.

### BRIEF DESCRIPTION OF THE FIGURES

FIG. 1 shows an HPLC chromatogram showing peak with a retention time of 7.63 minutes for pure, standard Betulinic acid.

FIG. 2 shows an HPLC chromatogram showing peak with a retention time of 7.67 minutes for the Betulinic acid of the herbal extract of this invention.

FIG. 3 is a NMR spectra of pure, standard Betulinic acid.

FIG. 4 is a NMR spectra of betulinic acid (major fraction) isolated from the

herbal extract of this invention.

FIG. 5 illustrates the effect of herbal extract on activity of protein kinase C in ovarian cancer cells (PA1).

FIG. 6 illustrates the effect of herbal extract on nucleosome release in ovarian cancer cells (PA1) in vitro.

FIG. 7 illustrates the effect of herbal extract on the levels of antiapoptotic protein bcl2 in ovarian cancer cells (PA1) in vitro.

FIG. 8 shows the percent reduction in VEGF levels in K562 cell line on treatment with herbal extract.

FIG. 9 shows the percent reduction in basic FGF levels K562 cell line on treatment with herbal extract.

FIG. 10 shows the endostatin levels on treatment of K562 cell line with herbal extract.

FIG. 11 shows the antitumor activity of the herbal extract upon early treatment of human colon xenografts.

FIG. 12 shows the antitumor activity of the herbal extract upon late treatment of human colon xenografts.

FIG. 13 shows the antitumor activity of the herbal extract upon early treatment of human melanoma xenografts.

FIG. 14 shows the antitumor activity of the herbal extract upon late treatment of human melanoma xenografts.

### DETAILED DESCRIPTION OF THE INVENTION

Accordingly, the invention provides a novel process for the preparation of a herbal extract of Ziziphus jubjuba rich in betulinic acid and having anticancer activity.

The invention provides a process for the preparation of herbal extract of Ziziphus jubjuba, said process comprising the steps of:

a) optionally drying the bark of Ziziphus jubjuba;

b) fragmenting/pulverizing the dried bark to reduce the size of bark pieces;

c) pre-macerating the bark in a solvent;

d) recovering the bark and optionally drying the bark;

e) macerating the bark again in a solvent;

f) filtering to recover the solvent; and

g) concentrating/heating to evaporate the solvent and obtain the extract.

In an embodiment of the invention, the solvent used in the pre-maceration step is a hydro-alcoholic solution. A hydro-alcoholic solution is a solution that contains less than 100% of alcohol, the remainder of the solution is water.

In another embodiment, the alcohol used is ethanol and in yet another embodiment the hydro-alcoholic solution contains 1 to 90 percent alcohol, more preferably 5 to 60 percent alcohol and still more preferably 10 to 50 percent alcohol.

In a further embodiment the bark treated with the hydro-alcoholic solution is optionally dried at 100° C. for 6-8 hours.

In still further embodiment, the bark is further macerated in (step e) with alcohol for 24 hrs.

In yet another embodiment, the bark macerated in alcohol (step e) is optionally sonicated or stirred to improve the extraction process.

In a further embodiment, the alcoholic extract is heated under vacuum to evaporate the solvent and obtain a dry extract.

In another feature, the process illustrated above gives quantitative extraction process from the bark. The preferred process comprises the following steps:

1) The dried outer bark of Zizyphus jujuba is crushed/pulverized and may be optionally dried.

2) The bark is then soaked in a hydro-alcoholic solution (pre-maceration step). The alcohol of preference being ethanol. The mixture is optionally stirred occasionally at room temperature for 12-20 hours. The hydro-alcoholic solution may contain 10-50% alcohol.

3) The above mixture is optionally sonicated to improve extraction and filtered or centrifuged to remove the liquid portion which is sent for recycling to recover the alcohol and the aqueous portion is discarded. The bark is optionally dried at 100° C. for 6-8 hours.

4) The treated bark of step-3 is then macerated using alcohol as the solvent for 12-20 hrs with optional sonication to improve extraction at room temperature.

5) The macerated mass is then filtered or centrifuged to separate the bark material from the mother liquor. The liquor part obtained is kept aside for further treatment.

6) The solid mass of the step 5) is washed with alcohol with optional sonication to improve extraction.

7) The solid mass is then separated and discarded, and, the alcoholic extract is mixed with the liquor part of the first macerate of the step-5.

8) The total alcoholic extract of the step-7 is then evaporated preferably under reduced pressure to obtain a dry mass of betulinic acid rich extract.

The processes described above can be used to prepare a herbal extract rich in betulinic acid from other species of Zizyphus including but not limited to Z. mauritiana, Z. rotundifolia, Z. mucronata, Z. nummularia, Z. lotus, Z. spina-christi, Z. obtusifolia.

The advantages of the process described above include:

1) The process yields an extract suitable for pharmaceutical use, the extract is rich in betulinic acid known to have anticancer activity. The extract provides betulinic acid that is easily solubilised as compared to pure isolated betulinic acid.

2) It is extremely simple, cost effective, highly economical and has commercial feasibility.

3) It does not involve any tedious process of chromatography at any stage as described in the prior art.

4) It does not involve any chemical reactions or conversion to any intermediate derivatives to facilitate extraction of the active ingredients.

5) It does not involve any energy intensive steps like heating or boiling.

6) The solvent used is a low boiling point solvent that can be easily recovered and recycled.

7) Depending upon the quality of the raw material which depends on seasonal collection, the yield of the main active principle i.e. betulinic acid varies from 0.3 to 1% w/w of the plant material.

8) Depending upon the quality of the raw material and the method of extraction used, the yield of the main active principle i.e. betulinic acid varies from 1 to 95% w/w preferably 10 to 70% w/w and more preferably 20 to 60% w/w of the herbal extract.

9) It is highly simple, economical, non-hazardous, eco-friendly, does not require any energy (heat) inputs and the solvent used is easily recyclable and recoverable.

The present invention also provides a composition comprising the betulinic acid rich extract of this invention, a derivative or salt thereof and a pharmaceutically acceptable carrier, diluent, or solvent. The composition may optionally and preferably contain pharmaceutically acceptable diluents, additives, filler, lubricant, excipients, solvents, binders, stabilizers, and the like. Such diluents may include: buffered saline, isotonic NaCl, Ringer's solution, water, distilled water, polyethylene glycol (neat or in water), 2% Tween in water, dimethyl-sulfoxide to 50% in water, propylene glycol (neat or in water), phosphate buffered saline, balanced salt solution, glycerol, and other conventional fluids that are suitable for intravenous administration. Pharmaceutical composition which provide from 500 mg to 5000 mg (preferably 1000 mg to 4000 mg) of the betulinic acid rich extract per unit dose are preferred and are conventionally prepared as tablets, lozenges, capsules, powders, aqueous or oily suspension, syrups, elixirs, and aqueous solutions. The nature of the pharmaceutical composition employed will, of course, depend on the desired route of administration.

The invention provides a method of treatment for humans, mammals, or other animals suffering from cancer or other tumors. The method may suitably comprise, consist of, or consist essentially of administering a therapeutically effective dose of the pharmaceutical composition so as to kill or inhibit the multiplication of cancer or tumor cells.

Preferably, the betulinic acid rich extract or a composition comprising the betulinic acid rich extract is used to treat or inhibit the growth of malignant tumors of the colon, intestine, stomach, breast, melanoma, glioblastoma, lung, cervix, ovary, prostate, oral cavity, larynx, liver, pancreas, kidney, bladder, or endothelial cells, or leukemia or myeloma.

The methods of this invention comprise, consist of, or consist essentially of administering systematically to the mammal a therapeutically effective dose of the herbal extract. An effective dose of herbal extract ranges from 10 mg/Kg. B. Wt to 200 mg/Kg. B. Wt (preferably 20-100 mg)/Kg. B. Wt) of the mammal, with the dose dependent inter alia on the effects sought, the manner of administration, the general health of the patient and the cancer being treated. Systemic administration refers to oral, rectal, nasal, transdermal, and parental (i.e., intramuscular, intravenous and subcutaneous). In accordance with good clinical practice, it is preferred to administer the composition at a dose that will produce anticancer effects without causing undue harmful side effects. The composition may be administered either alone or as a mixture with other therapeutic agents such as 5-fluorouracil, methotrexate, etoposide, paclitaxel, taxotere, doxorubicin, daunarubicin, vincristine, vinblastine and other such known and established anticancer drugs.

An effective amount means that amount of a drug or pharmaceutical agent that will elicit the biological or medical response that is being sought. In a preferred embodiment, the effective amount of betulinic acid is not less than 10% w/w of the extract.

A few illustrative compositions of the herbal extract are given below.

Composition-1

Emulsion compositions for the said purpose are given below:

| | | |
|---|---|---|
| Herbal extract | 0.5-15% | w/v |
| Arlamol | 4.5% | v/v |
| Polysorbate 80 | 3.5% | w/v |
| Absolute alcohol | 2.0 | v/v |
| Sodium Benzoate | 0.5% | w/v |
| Flavour | 1.0% | v/v |
| Purified water | up to 100 | v/v. |
| Composition-2 | | |
| Herbal extract | 0.5-25% | w/v |
| Cottonseed oil | 3-5% | v/v |
| Polyoxyethylene 20 Sorbitan Monostearate | 2-5% | v/v |
| Sodium benzoate | 0.5% | w/v |
| Absolute alcohol | 2% | v/v |
| Purified water | upto 100% | v/v |
| Flavour | 0.8% | v/v |
| Composition-3 | | |
| Herbal extract | 0.5-15% | w/v |
| Soyabean oil | 5.5% | v/v |
| Polysorbate 80 | 3.5% | w/v |
| Absolute alcohol | 2.0% | v/v |
| Purified Water | upto 100% | v/v |
| Flavour | qs | v/v |

A suspension composition for the said purpose is given below:

Composition-4

| | | |
|---|---|---|
| Herbal Extract | 5-40% | w/v |
| Polysorbate 80 | 0.05-0.5% | w/v |
| Xanthan Gum | 0.1-0.2% | w/v |
| Sodium chloride | 0.5% | w/v |
| Parabens | 0.2% | w/v |
| Propylene Glycol | 2% | v/v |
| Sugar Syrup to make | 100% | v/v |

A Capsule composition for the said purpose is given below:

| Composition-5 | | |
|---|---|---|
| Herbal Extract | 1-5% | w/w |
| Polysorbate 80 | 0.01-0.05% | w/w |
| Sodium Starch Glycolate | 5-10% | w/w |
| Starch | 10-30% | w/w |
| Microcrystalline Cellulose | 30-50% | w/w |
| Lactose | 20-50% | w/w |
| Composition-6 | | |
| Herbal Extract | 1-5% | w/w |
| Lactose | 10-25% | w/w |
| Polyvinylpyrrolidone | 5-10% | w/w |
| Starch | 5-10% | w/w |
| Talc | 2-5% | w/w |
| Magnesium Stearate | 30-50% | w/w |

It may be noted that the above compositions are provided for illustrations of the preferred embodiment only and should not be construed to limit the spirit or scope of the present invention in any way.

The above process is described in detail by the following examples, which are provided for illustrating only, and should not be construed to limit the scope of the present invention.

### EXAMPLE 1

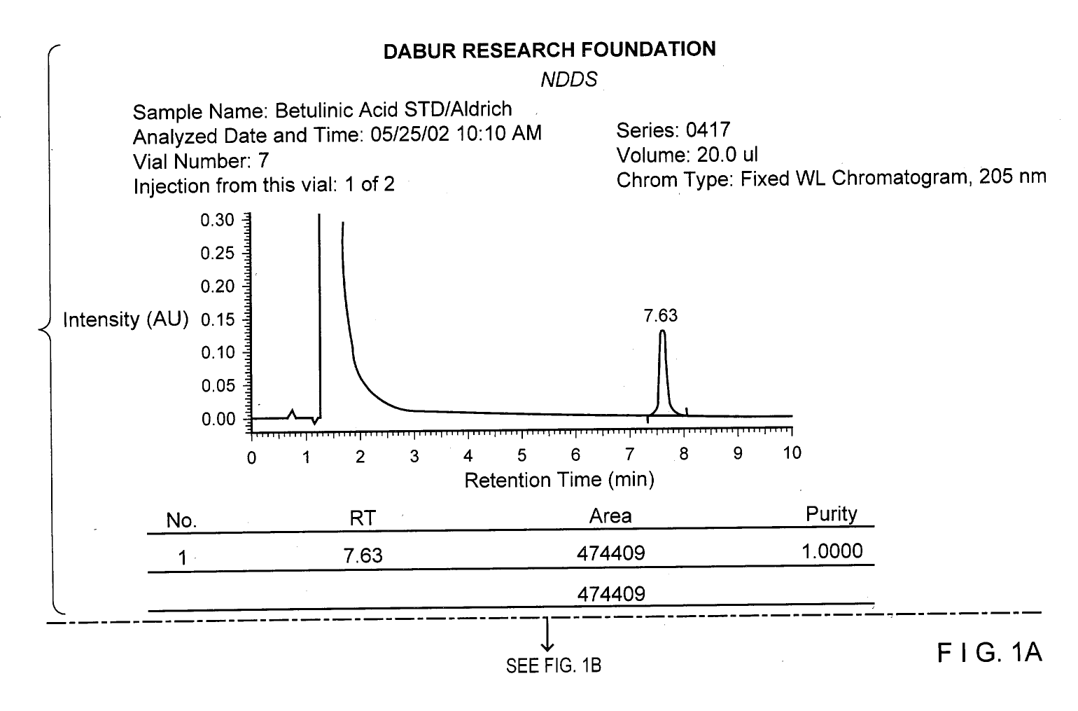

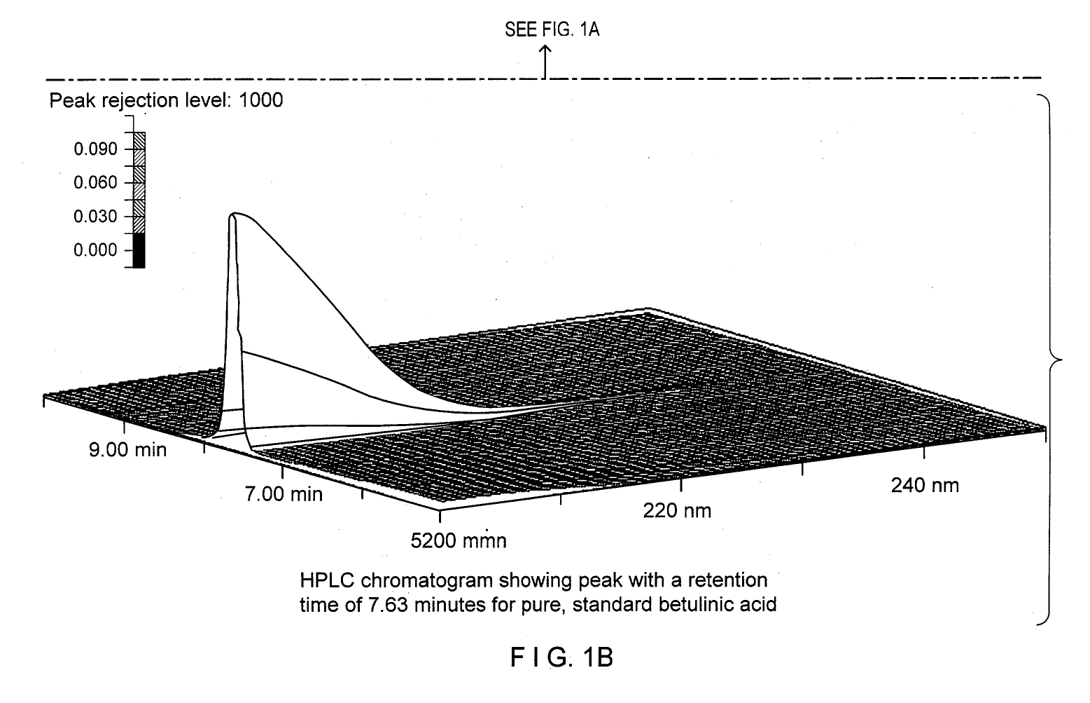

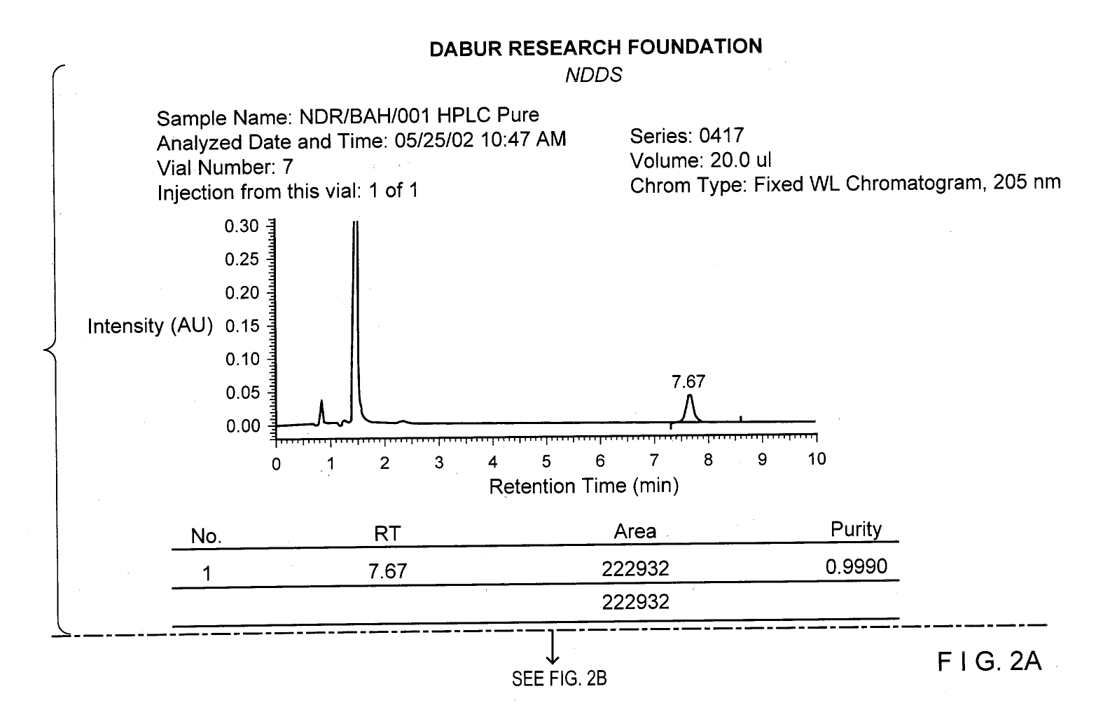

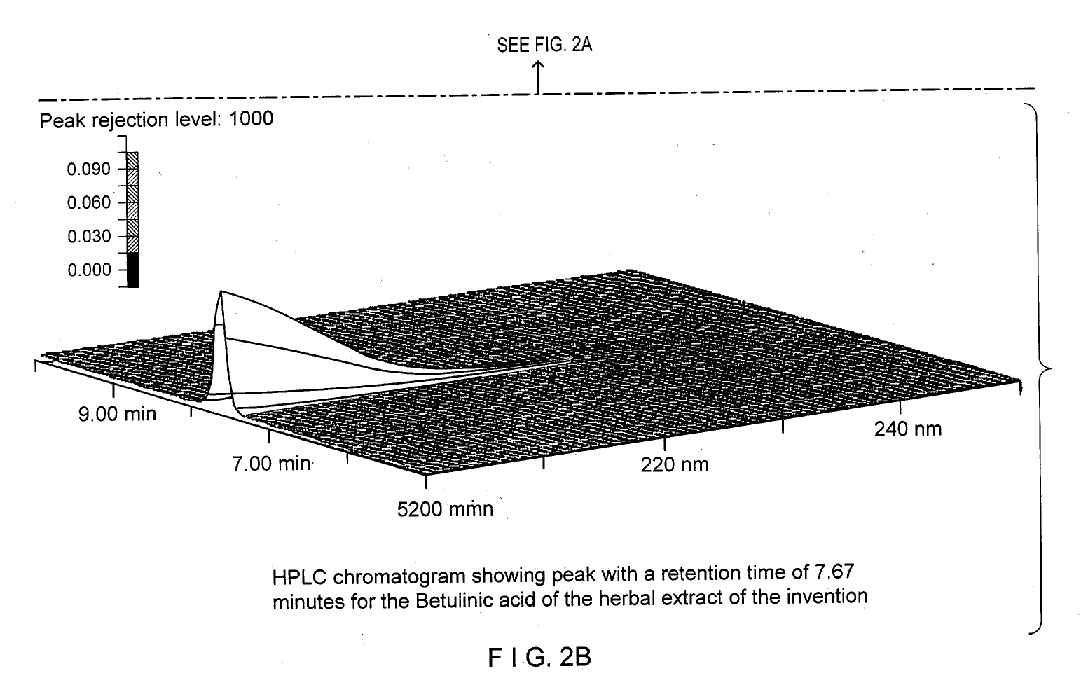

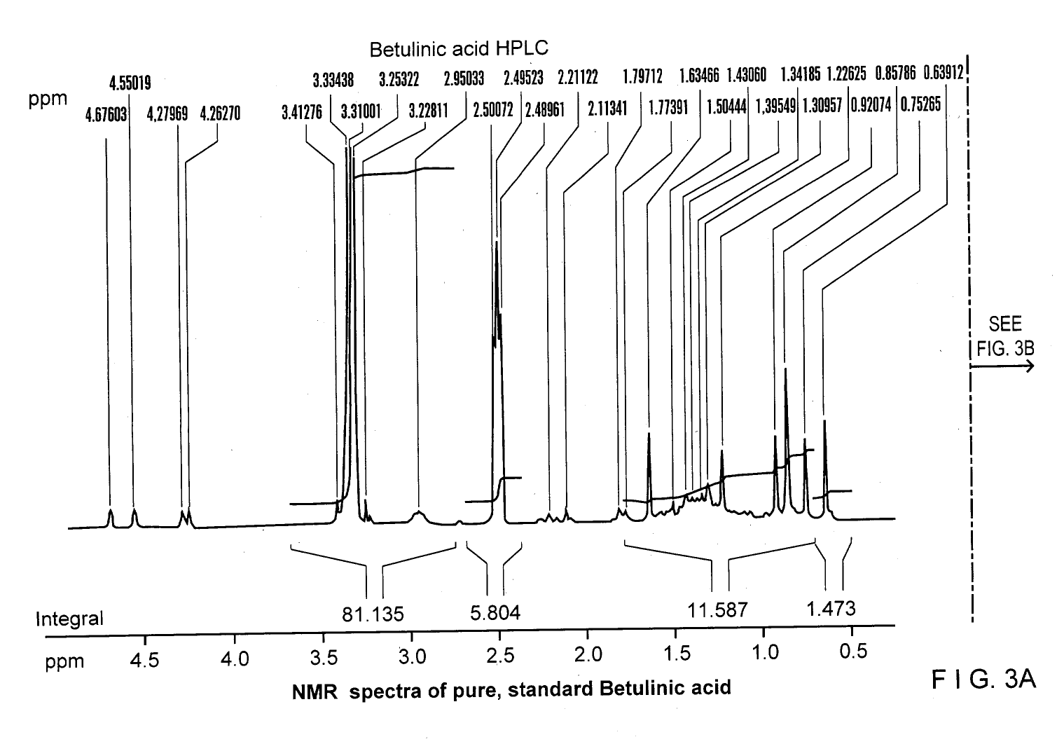

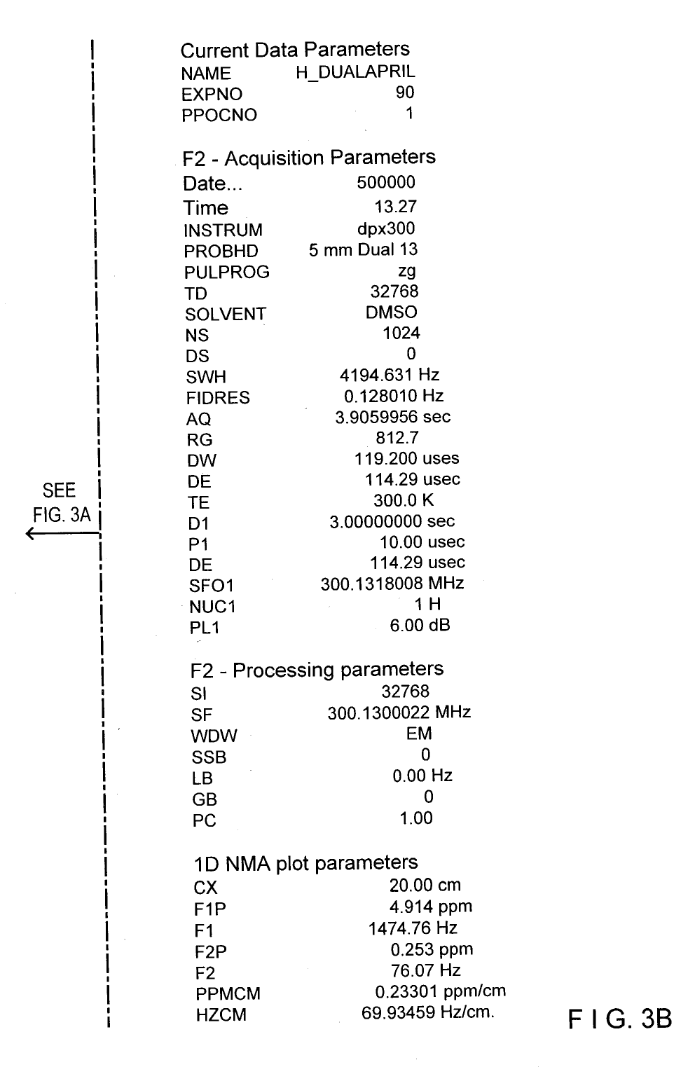

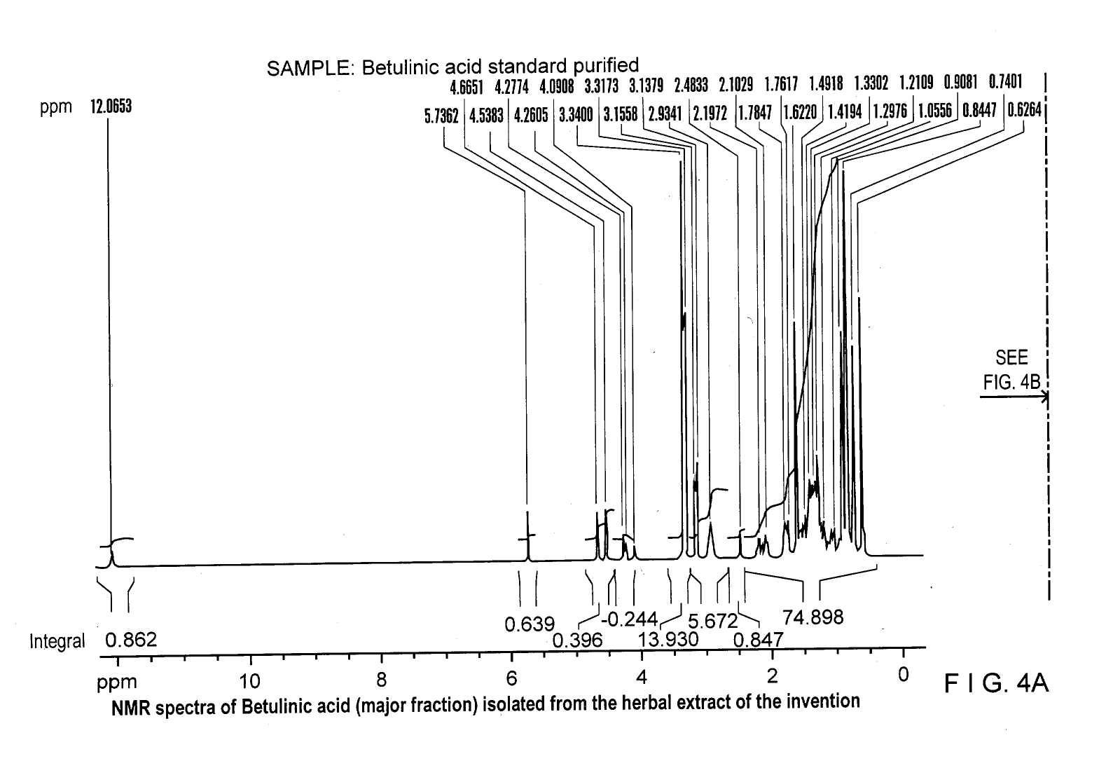

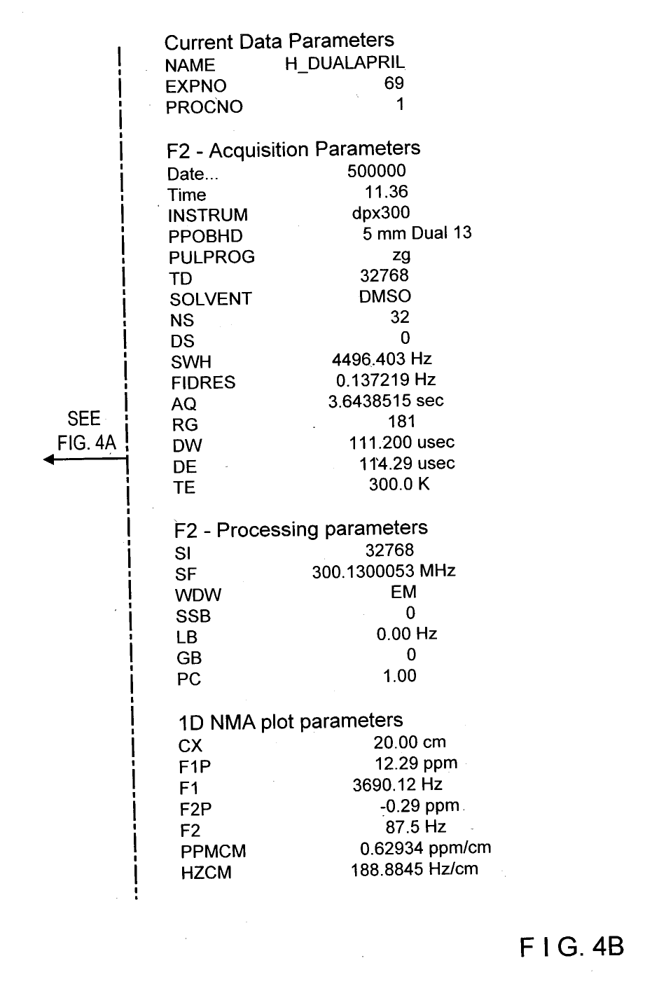

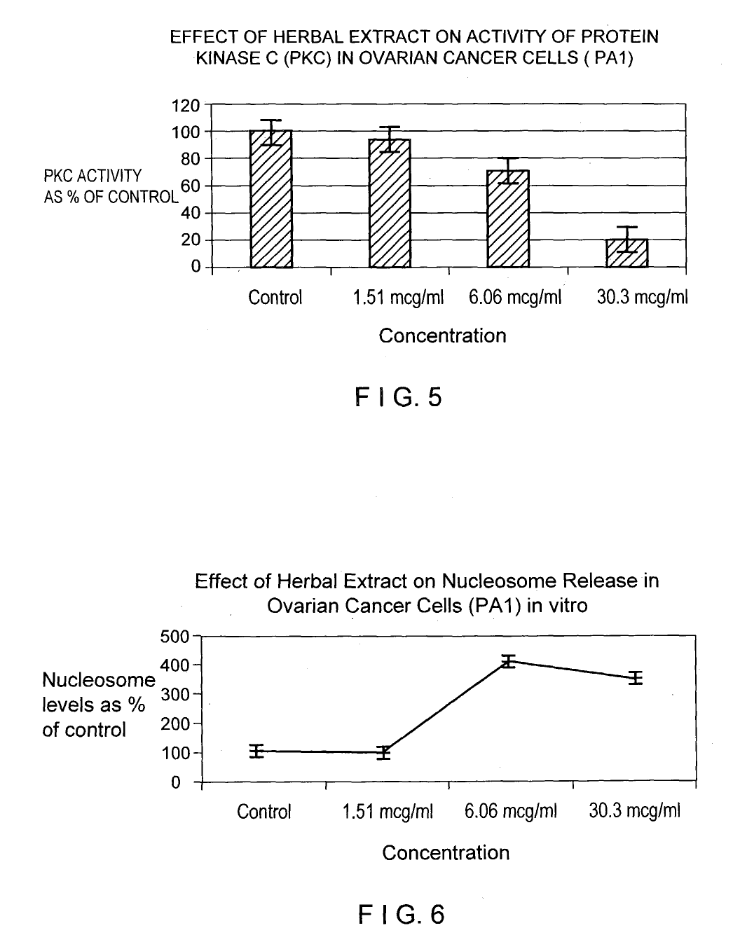

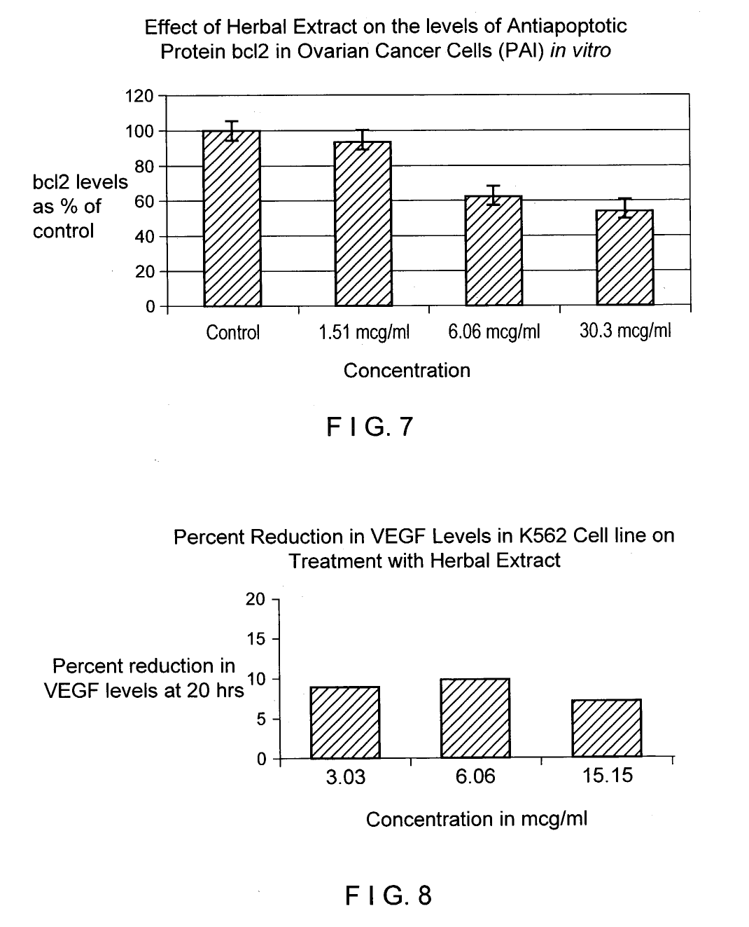

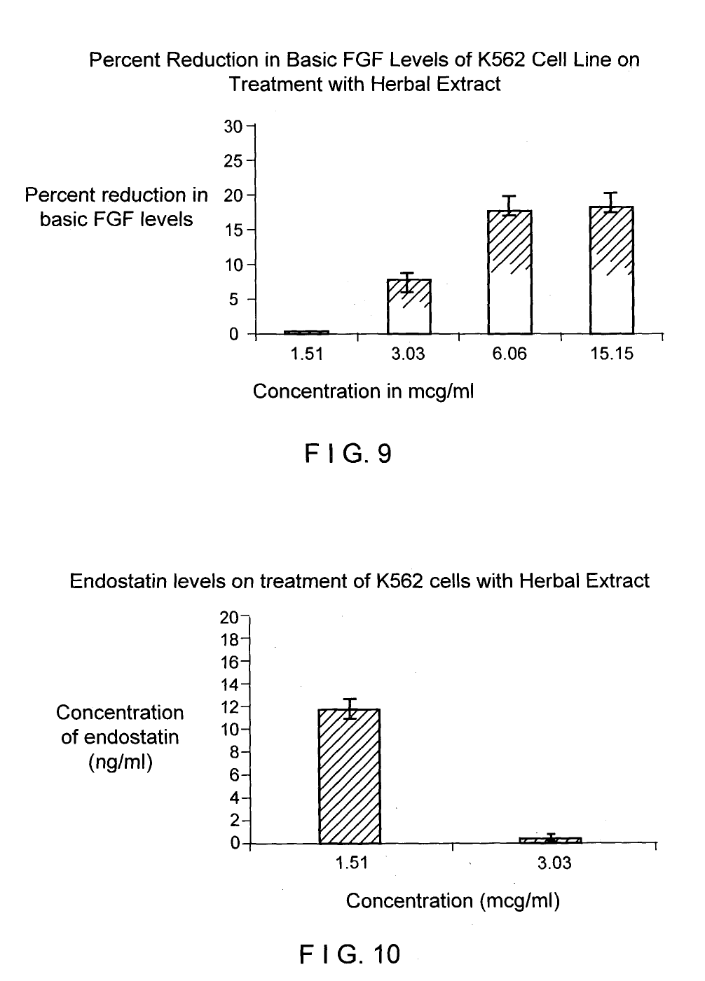

FIG. 1 shows a chromatogram and Diode Array Detector Profile of pure, standard Betulinic acid procured from M/s Aldrich. Pure betulinic acid has a retention time (RT) of 7.63 minutes. FIG. 2 shows under same set of conditions the chromatogram and Diode Array Detector Profile of the betulinic acid isolated from the herbal extract which also shows a retention time of 7.67 minutes. This confirms that the major constitute isolated from the extract is betulinic acid. FIGS. 3 and 4 give the NMR spectra of Standard Betulinic Acid and Betulinic acid (major fraction) isolated from the herbal extract respectively.

### EXAMPLE 2

The bark of Zizyphus jujuba optionally dried at about 60° C. is ground/pulverised (#40-60 mesh). The ground bark (1000 g) is then soaked in about 4 litres of hydro-alcoholic solution (20%) at room temperature for 18 hours with occasional stirring. At the end of 18 hours the mixture is sonicated for about an hour. The solid part is then separated by filtration or centrifugation and optionally dried at 100° C. for 6 to 8 hours. The liquid part is kept aside for recovering the alcohol. The treated bark is then macerated using alcohol (4 litres) as the solvent for about 18 hours and optionally sonicated for 1 hour at room temperature. The macerated mass is then filtered through a #200 mesh, the solid mass retained on the mesh is washed with another 1 litre of alcohol, shaken for about 10-15 minutes and combined with the first filtrate. The solid mass is then rejected. The combined filtrate or the alcoholic extract is then evaporated under reduced pressure to obtain the dried herbal extract.

### EXAMPLE 3

Herbal extract was tested for cytotoxicity against 23 human cancer cell lines. Briefly, a three day MTT cytotoxicity assay was performed, which is based on the principle of uptake of MTT (3-(4,5-dimethylthiazol-2-yl)-2,5-diphenyl tetrazolium bromide), a tetrazolium salt, by the metabolically active cells where it is metabolized by active mitochondria into a blue colored formazan product that is read spectrophotometrically. MTT was dissolved in phosphate buffered saline (pH 7.4) to obtain an MTT concentration of 5 mg/ml; the resulting mixture was filtered through a 0.22 μm filter to sterilize and remove a small amount of insoluble residue. Herbal extract was dissolved in 2.5% DMSO and DMEM. The control cells were treated with 2.5% DMSO in DMEM. All experiments were carried out in triplicates. For each type of cancer cell, 20,000 to 50,000 cells were seeded in a 96-well culture plate and incubated with herbal extract in a CO₂ incubator for 72 hours. The final concentration range of herbal extract was 5 to 200 μg/ml. Control cells not treated with herbal extract were similarly incubated. The assay was terminated after 72 hours by adding 100 μug of MTT to each well, then incubating for additional one hour, and finally adding 50 ul of 10% SDS-0.01N HCl to each well to lyse the cells and dissolve formazan. After incubating for one hour, the plate was read spectrophotometrically at 540 nm and the percentage cytotoxicity calculated. Table (1) shows the ED₅₀ values of in vitro cytotoxicity of herbal extract on various human cancer cell lines. The ED₅₀ values of the observed cytotoxicity of the herbal extract in different cell lines varied from 9 to 80 μg/ml. The betulinic acid content in herbal extract is 33.3%, hence the ED₅₀ values corrected for the betulinic acid content ranges from 1.57-24 μg/ml.

TABLE (1)
Cyotoxicity Profile of Herbal Extract

| | | | ED₅₀ (μg/ml) of |
|---|---|---|---|
| | | | herbal extract |
| | | ED₅₀ (μg/ml) | (corrected for |
| | | of | Betulinic acid |
| S.No | Cell line | herbal extract | content) |
| | Leukemia & Myeloma | | |
| 1 | K 562 (Human myelogenous | 15.5 ± 3.5 | 4.5 ± 1.6 |
| | leukemia) | | |
| 2 | MOLT-4 (Human lymphoblastic | 29.0 ± 3.0 | 8.78 ± 2.5 |
| | leukemia) | | |
| 3 | RPMI 8226 (Myeloma) | 56.0 ± 4.5 | 16.96 ± 3.5 |
| | Gastro-intestinal cancers | | |
| 4 | HT29 (Colon) | 5.2 ± 0.7 | 1.57 ± 0.34 |
| 5 | PTC (colon) | 75.0 ± 5.0 | 22.7 ± 4.5 |
| 6 | Int.407 (Intestine) | 9.0 ± 0.45 | 2.7 ± 1.7 |
| 7 | HuTu80 (Stomach) | 30.0 ± 2.5 | 9.0 ± 3.0 |
| | Breast cancer | | |
| 8 | HBL100 (Breast) | 36.5 ± 3.6 | 11.06 ± 2.5 |
| 9 | MCF-7 (Breast) | 80.0 ± 5.9 | 24.2 ± 3.5 |
| | Melanoma | | |
| 10 | SKMEL28 (Melanoma) | 52.0 ± 4.5 | 16 ± 1.5 |
| 11 | B16F10 (Murine melanoma) | 35.0 ± 4.5 | 10.6 ± 2.5 |
| | CNS cancers | | |
| 12 | U87MG (Human glioblastoma) | 63.0 ± 4.6 | 19.0 ± 2.5 |
| | Lung cancer | | |
| 13 | L132 (lung) | 65.0 ± 5.6 | 19.6 ± 3.1 |
| | Cervical cancer | | |
| 14 | SiHa (Cervix) | 55.0 ± 4.6 | 16.6 ± 2.5 |
| | Ovarian cancer | | |
| 15 | PA-1 (Human ovary) | 22.0 ± 1.8 | 6.6 ± 2.4 |
| | Prostate cancer | | |
| 16 | DU145 (Prostate) | 40.0 ± 2.5 | 12.1 ± 3.4 |
| | Oral & Laryngeal cancers | | |
| 17 | KB (Oral) | 220 ± 1.6 | 6.6 ± 0.7 |
| 18 | HeP.2 (Larynx) | 42.0 ± 3.5 | 12.7 ± 2.1 |
| | Liver cancer | | |
| 19 | HepG2 (Liver) | 19.0 ± 2.0 | 5.75 ± 0.75 |
| | Pancreatic cancer | | |
| 20 | MiaPaCa2 (Human pancreas) | 60.0 ± 3.6 | 18.18 ± 2.0 |
| | Urogenital cancer | | |
| 21 | 293 (Kidney) | 25.0 ± 2.5 | 7.5 ± 1.6 |
| 22 | T-24 (Bladder) | 56.0 ± 3.5 | 16.9 ± 2.5 |
| | Transformed Endothelial cells | | |
| 23 | ECV304 (Endothelial) | 50.0 ± 3.5 | 15.15 ± 1.7 |

### EXAMPLE 4

### Effect of Herbal Extract on the Activity of Protein Kinase C (PKC)

### In Ovarian Cancer Cells (PA1) In-vitro

Protein kinase C (PKC) is a family of closely related lipid dependent & diacylglycerol activated isoenzymes, with an important role in mitogenesis and tumor promotion. Sustained activation of PKC activity in-vivo plays a critical role in regulation of proliferation and tumorigenesis. The assay for quantitation of the activity of PKC is based on the enzyme linked immunosorbent assay (ELISA) that utilizes a synthetic PKC pseudosubstrate & a monoclonal antibody that recognizes the phosphorylated form of the peptide.

The effect of herbal extract on the activity of Protein Kinase C (PKC) was quantitated by an enzyme-linked immunosorbent assay (ELISA) as per the manufacturers instructions. Briefly 1.0×10⁶ of human ovarian cancer cells (PA1) were plated per well in 6-well tissue culture plates in 2 ml of DMEM containing 10% Fetal calf serum. The cells were incubated overnight at 37° C. and 5% CO₂. The medium was changed to DMEM without serum. Herbal extract was then dissolved in 2.5% DMSO in DMEM. The cells were incubated with the herbal extract at final concentration ranging from of 5-200 μg/ml for a period of 20 minutes at 37° C. in 5% CO₂. The control cells were treated with 2.5% DMSO in DMEM. All experiments were carried out in triplicates. The medium containing the herbal extract was aspirated, and the cells were washed with Phosphate buffered saline (PBS, 50 mM, pH 7.2) twice. The cells were scraped with sterile cell scrapers, and spun at 207×g for 5 minutes. The cell pellet was dissolved in 1 ml of ice-cold sample preparation buffer (50 mM Tris-HCl, 50 mM 2-mercaptoethanol, 10 mM EGTA, 5 mM EDTA, 1 mM PMSF, 10 mM Benzamidine, pH 7.5). The cell pellets were sonicated four to five times, each with time interval of 5-10 seconds. The cells were centrifuged at 1,000,00×g for a period of 60 minutes at 4° C. The supernatant was aspirated from each set of experiments. The reaction mixture for quantitation of the PKC activity contained 25 mM Tris-HCl (pH7.0), 3 mM MgCl₂, 0.1 mM ATP, 2 mM CaCl₂, 50 μg/ml Phosphatidylserine, 0.5 mM EDTA, 1 mM EGTA, and 5 mM 2-mercaptoethanol. The reaction mixture was preincubated for 5 minutes at 25° C. 50 μg of the cell lysate from different experiments was added to the individual reaction mix and added to microtitre plates coated with the substrate for PKC. The plates were incubated for 15 minutes at 25° C. in a water bath. The PKC mediated reaction was stopped by the addition of 100 μl of stop solution. The plates were washed 5 times with the wash solution, and 100 μl of the biotinylated antibody to the phosphorylated substrate was added per well. The plates were incubated at 25° C. for 60 minutes. The plates were washed 5 times and 100 μl of peroxidase conjugated streptavidin was added to each well. The plates were incubated at 25° C. for 60 minutes. The plates were washed five times and 100 μl of substrate was added to each well. The plates were incubated at 25° C. for 3-4 minutes, and the reaction was stopped by the addition of stop solution. The optical density was read at 492 nm, and the PKC activity expressed as percent of that in control cells.

FIG. (5) shows the data on modulation of PKC activity by herbal extract in ovarian cancer cells. Data in FIG. (5) is reported after correction for betulinic acid (refer table 1) content in the herbal extract. As seen below herbal extract inhibits the PKC activity in a dose dependent manner in human ovarian cancer cells in-vitro, within 20 minutes of treatment. The data suggests that herbal extract inhibits PKC activity, which in turn may be modulating the Ras-MAPK pathway to bring about the anticancer effects.

### EXAMPLE 5

### Effect of Herbal Extract on Free Nucleosome Induction in Cancer Cells In-vitro

The levels of free nucleosomes were quantitated as per the instructions detailed for the Nucleosome ELISA (Oncogene Research Products). Briefly 1×10⁶ human ovarian cancer cells (PA1) were plated per well in DMEM containing 10% FCS in 6 well sterile tissue culture plates. The cells were incubated overnight to allow complete attachment of the cells. Herbal extract was dissolved in 2.5% DMSO in DMEM. The cells were incubated with concentrations of herbal extract varying from 5-200 μg/ml for a period of 6 hours. The control cells were treated with 2.5% DMSO & DMEM alone. All experiments were carried out in triplicates. The cells were lysed & the protein was estimated in the Bicinchoninic acid based assay. The lysates were frozen at −20° C. for a period of 18 hours prior to quantitation. The lysates were diluted 1:1 in the sample diluent. The standard nucleosome was dissolved & 100 μl of the cell lysates and the standards in different concentrations was added in duplicate in microtitre plates coated with appropriate DNA binding proteins. The plates were incubated for 3 hours at room temperature. The wells were washed with wash buffer thrice and 100 μl of anti-Histone 3 biotinylated antibody was added per well & incubated at room temperature for a period of one hour. The wells were washed with the wash buffer thrice, & 100 μl of the streptavidin conjugate diluted 1:400 was added per well. The wells were washed thrice in wash buffer, and 100 μl of the substrate was added per well & the plates were incubated in dark for 30 minutes at room temperature. The reaction was stopped and the optical density of the wells was read at dual wavelengths of 450/595 nm. As shown in FIG. (6), the free nucleosomes increased significantly in response to the treatment with herbal extract for a period of 6 hours. Data in FIG. (6) is reported after correction for betulinic acid (refer table 1) content in the herbal extract

### EXAMPLE 6

### Effect of Herbal Extract on Anti-apoptotic Protein Bcl2 in Ovarian Cancer Cells (PA1) In-vitro

Bcl-2 was quantitated by sandwich enzyme immunoassay (Oncogene Research products, Cat no. QIA23) according to the listed instructions. Briefly 2.5×10⁶ Human Ovarian cancer cells (PA1) were plated per well in DMEM with 10% FCS in 6 well sterile tissue culture plates. The cells were incubated for 12 hours to allow complete attachment to the wells. herbal extract was dissolved in 2.5% DMSO in DMEM. The media was changed to sera free DMEM and the cells were incubated with herbal extract in concentrations ranging from 5-100 μg/ml. The control cells were treated with 2.5% DMSO in DMEM for 20 hours. All experiments were carried out in triplicates. The supematent was removed and the cells scraped and lysed at 4° C. to extract the Bcl-2 protein. Protein was estimated by Bicinchonic acid based assay. The cell lysates were diluted 1:1 in sample diluent. 50 μl of the cell lysates & nucleosome standards were added per well, in microtitre plates coated with monoclonal antibody to bcl2. 50 μl of the detector antibody was added per well. & the plates were incubated for a period of 2 hours. The wells were washed thrice, & 100 μl of the conjugate diluted 1:200 was added to all the wells. The wells were washed thrice, & 100 μl of the substrate solution was added per well. The absorbance was read at dual wavelengths of 450/595 nm. As shown in FIG. (7) below herbal extract inhibits bcl2 levels in ovarian cancer cells (PA1) in a dose dependent manner in-vitro, suggesting that it is providing the necessary signal for induction of programmed cell death. Data in FIG. (7) is reported after correction for betulinic acid (refer table 1) content in the herbal extract

### EXAMPLE 7

### Antiangiogenic Activity of Herbal Extract

The antiangiogenic potential of herbal extract was determined by measuring its effect on endothelial cell proliferation. Herbal extract had significant cytotoxicity on endothelial cells (ECV304). We then looked for the effect on the levels of three known markers of angiogenesis in cell supernatants of human tumor cells. These markers are (i) Vascular Endothelial Growth Factor (VEGF), (ii) basic Fibroblast Growth Factor (bFGF), and (iii) Endostatin. While VEGF and bFGF are pro-angiogeneic, Endostatin has angiogenesis inhibitory activity. Hence, decrease in the levels of VEGF and bFGF and increase in the levels of Endostatin are indicative of antiangiogenic activity.

The above-mentioned markers were assayed in cell supernatants after treating the cells with previously determined non-cytotoxic concentrations of herbal extract for a predetermined time. The estimations were performed using commercially available ELISA kits. The assay employs the quantitative sandwich enzyme immunoassay technique. A monoclonal antibody specific for VEGF, bFGF or Endostatin respectively has been pre-coated onto a microplate. Standards and samples are pipetted into the wells and any VEGF, bFGF or Endostatin respectively present is bound by the immobilized antibody. After washing away any unbound substances, an enzyme-linked polyclonal antibody specific for the respective protein is added to the wells. Following a wash to remove any unbound antibody-enzyme reagent, a substrate solution is added to the wells and color develops in proportion to the amount of protein bound in the initial step. The color development is stopped and the intensity of the color is measured spectrophotometrically.

a. Inhibition of Vascular Endothelial Growth Factor by Herbal Extract

K562 cells were plated at the density of 8-10×10⁵ cells in RPMI 1640 media in a six well plate. After an overnight incubation of cells at 37° C., herbal extract was added to the treated wells at previously determined non-cytotoxic concentrations. The untreated wells acted as controls. The plate was incubated at 37° C. in CO₂ incubator. Supernatant was collected from treated and untreated wells after 6 hours and 20 hours and spun down at 250×g for 10 minutes to remove any cellular material and stored at −20° C. till further use. The levels of VEGF in the supernatant were quantified using Human VEGF Immunoassay (Quantikine, R&D Systems, catalogue No. DVE00) kit by following manufacturer's instructions.

Herbal extract causes a 10% inhibition in secretion of VEGF from K562 cells at 6 hours. Data in FIG. (8) is reported after correction for betulinic acid (refer table 1) content in the herbal extract.

b. Inhibition of Basic Fibroblast Growth Factor by Herbal Extract

K562 cells were plated at the density of 8-10×10⁵ cells in RPMI 1640 media in a six well plate. After an overnight incubation of cells at 37° C., herbal extract was added to the treated wells at previously determined non cytotoxic concentrations. The untreated wells acted as controls. The plate was incubated at 37° C. in CO₂ incubator. Media was collected from treated and untreated wells after 6 hours and 20 hours and spun down at 250×g for 10 minutes to remove any cellular material and stored at −20° C. till further use. The levels of FGF basic in the supernatant were quantified using Human FGF basic Immunoassay (Quantikine, R&D Systems, catalogue No. DFB50) kit by following manufacturer's instructions.

Herbal extract causes a 18.4% inhibition in secretion of bFGF from K562 cells at 6 hours (FIG.-9). Data in FIG. (9) is reported after correction for betulinic acid (refer table 1) content in the herbal extract.

c. Increase in Endostatin Levels by Herbal Extract

K 562 cells were plated at the density of 8-10×10⁵ cells in RPMI 1640 media in a six well plate. After an overnight incubation of cells at 37° C., the drug was added to the treated wells at non cytotoxic concentrations. The untreated wells acted as controls. The plate was incubated at 37° C. in CO₂ incubator. Media was collected from treated and untreated wells after 6 hours and 20 hours and spun down at 250×g for 10 minutes to remove any cellular material and stored at −20° C. till further use. The levels of Endostatin in the supernatant were quantified using Human Endostatin Protein Accucyte enzyme immunoassay (Oncogene Research Products, Cat# QIA65) kit by following manufacturer's instructions.

The herbal extract causes an increase in secretion of Endostatin from non-detectable basal levels to 12 ng/ml in K562 cells at 20 hours (FIG.-10). Data in FIG. (10) is reported after correction for betulinic acid (refer table 1) content in the herbal extract The antiangiogenic effect could be a cumulative effect of reduction in proliferation of endothelial cells and modulation of the related growth factors.

### EXAMPLE 8

### The Anti-tumor Activity of Herbal Extract on Human Tumor Xenografts Grown in Nude Mice

Human Tumor Xenografts were initiated in Balb/c athymic mice by subcutaneous inoculation of a single cell suspension of PTC (Primary tumor cells of colon adenocarcinoma) or B16F10 (Murine melanoma cells) tumor cells. Herbal extract was prepared as described before and reconstituted fresh in saline before administration to the animals at a concentration of 5 mg/ml. A dose of 100 mg/kg Body weight was administered to mice on alternate days for at least 3 weeks. Two different routes Oral route and Intra-peritoneal route and two different treatment schedules (Early treatment—24 hrs after tumor cell inoculation and Late treatment—When tumor volume reaches 300-600 cu.mm) were tested. Tumor-bearing mice were randomly divided into groups of 3 animals each. Animals not receiving treatment served as control. Oral dosing was done by gastric feeding using oral gavage tubes inserted through the mouth into the stomach or lower esophagus to ensure administration of the compound into the stomach. Intra-peritoneal dosing was done using 23 G needle and test substance administered into the peritoneal cavity. The anti-tumor activity was monitored by measuring tumor volumes every fourth day and calculating volumes using the formula 0.4×W²×L (W=smaller dia, L=larger dia,).

Table-2 shows Percent tumor regression caused by the test compound in different experiments.

TABLE 2

| | |
|---|---|
| Group | Tumor Regression |

PTC (Colon) Tumor regression on day 19 post treatment

| | |
|---|---|
| Early treatment | |
| PTC (ORAL) | 73.3% |
| PTC (I.P.) | 73.3% |
| Late treatment | |
| PTC (ORAL) | 31.7% |
| PTC (I.P.) | 60.1% |

B16F10 (Melanoma) Tumor regression on day 26 post treatment

| | |
|---|---|
| Early treatment | |
| B16F10 (ORAL) | 57.3% |
| B16F10 (I.P.) | 73.6% |
| Late treatment | |
| B16F10 (ORAL) | 53.7% |
| B16F10 (I.P.) | 64.3% |

Percent tumor regression is calculated as follows:

$$\frac{\begin{matrix}Tumor volume ( untreated , cu . mm ) - \\ Tumor volume ( treated , cu . mm )\end{matrix}}{Tumor volume ( untreated , cu . mm )} \times 100$$

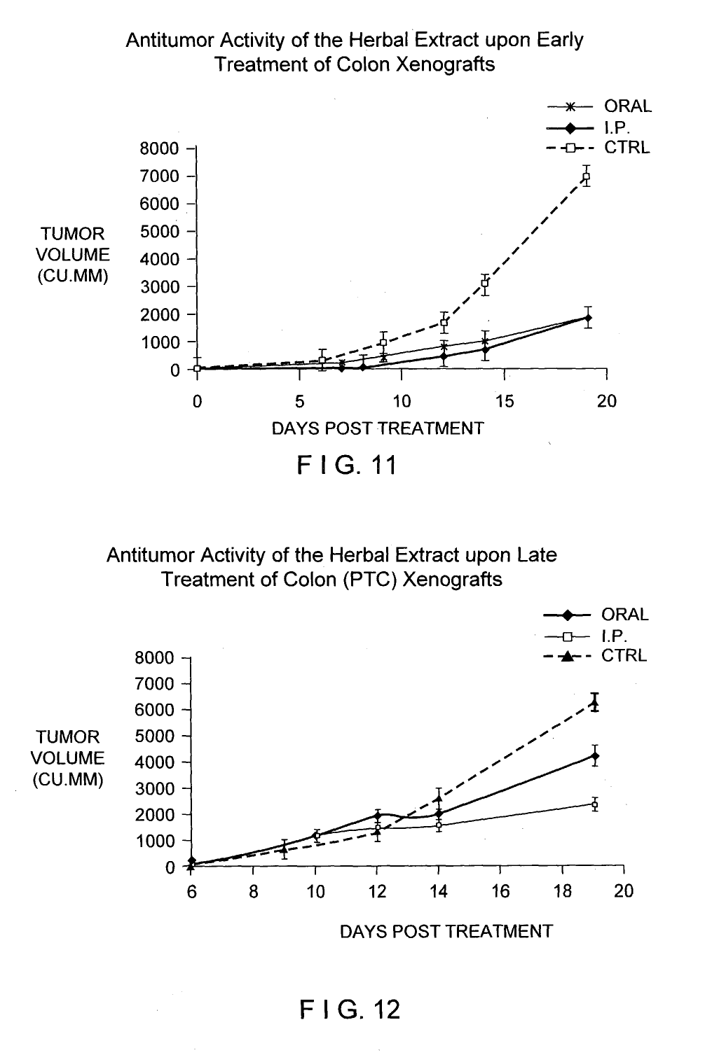

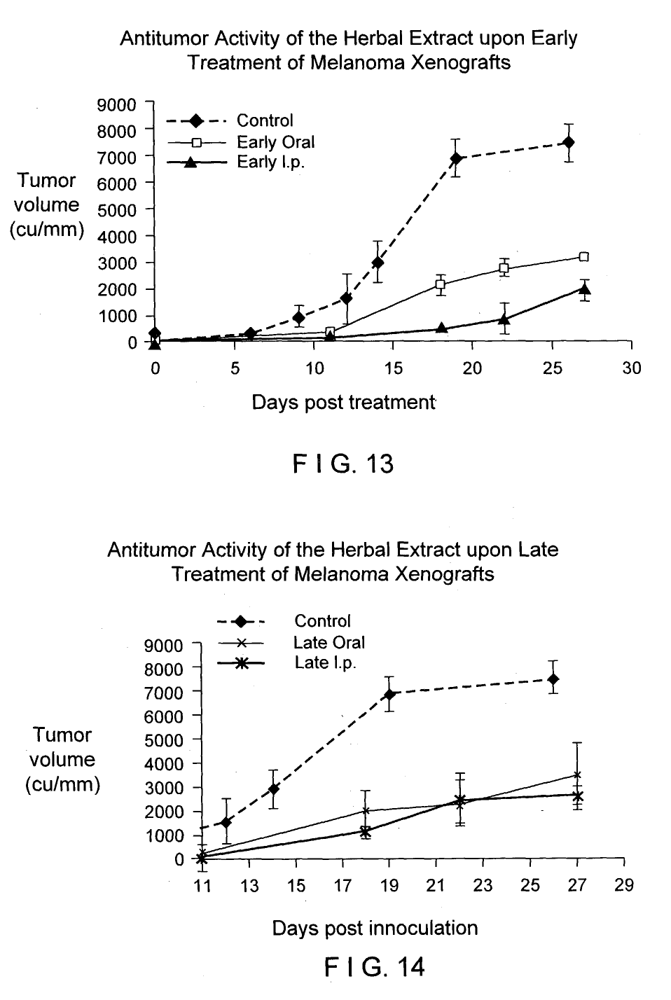

Tumor growth curves (FIGS. 11 to 14) show the pattern of tumor growth in treated and untreated (control) animals in different experiments.

### EXAMPLE 9

### Toxicity of Herbal Extract in Mice

### Acute Toxicity Studies

Single dose acute toxicity studies were carried out to determine the median lethal dose (LD₅₀) in Albino mice (Mus musculus) using two different routes of administration—oral and intraperitoneal. 5 doses were given to elicit a range of response to treatment in a batch of 5 male and 5 female animals per dose. The control group of animals were similarly treated with diluent only. All groups of animals were observed for a period of 15 days. All gross visible toxic signs and symptoms were recorded and the animals were subjected to necropsy. All the findings not considered normal were recorded.

The acute LD₅₀ values obtained were as follows:

Acute LD₅₀ in mice (Oral), Dose X=100 mg/kg

Male: >1.6 g/kg

Female: >1.6 g/kg

Acute LD₅₀ in mice (I.P.), Dose X=100 mg/kg

Male: >1.6 g/kg

Female: >1.6 g/kg

No mortality was recorded even at concentrations of 1.6 g/kg body weight of the extract or 16 times the therapeutic dose of 100 mg/kg. There were no apparent toxic signs or symptoms and no significant loss of body weight during the period of observation. Hence the acute toxicity dose is greater than the highest dose tested.

### Sub-acute Toxicity Studies

Sub-acute studies were designed to evaluate the toxicological effects of herbal extract when administered by two routes—oral and intra-peritoneal in mice daily for 15 days.

3 dose levels of herbal extract equivalent to 100 mg/kg, 200 mg/kg, 400 mg/kg of Betulinic acid for oral dosing and a sterile preparation of herbal extract equivalent to 50 mg/kg, 100 mg/kg, 200 mg/kg of Betulinic acid for intra-peritoneal dosing were given to elicit a range of response to treatment in order to assess the toxicity of herbal extract.

### Oral Dosing

No mortality was recorded in any of the doses tested. Food and water consumption were normal, no marked difference in the mean body weights was seen between treated and untreated groups, there was no alopecia and no other apparent toxic signs or symptoms were observed during the course of the study. The mean organ weights of different groups of animals were within normal limits except for slight enlargement of spleen of treated groups of animals in all doses tested as compared to untreated group of animals. Terminal hematology and blood biochemistry values were within normal limits except for a dose-related increase in the Total WBC count.

Under the conditions of the study, 15 days oral administration of herbal extract in albino mice at the dosage level containing 400 mg/kg Betulinic acid did not produce any observable toxic effects when compared to untreated group of animals. Hence may be considered as No Observable Effect Level.

N.O.E.L (oral)=400 mg/kg

### Intra-peritoneal Dosing

No mortality was recorded in any of the doses tested. All doses caused mild inflammation at the site of injection. Food and water consumption were normal, no marked difference in the mean body weights was seen between treated and untreated groups. There was no alopecia and no other apparent toxic signs or symptoms were observed during the course of the study. The mean organ weights of different groups of animals were within normal limits of both Vehicle and herbal extract treated groups of animals as compared to untreated group of animals. Terminal hematology and blood biochemistry values of herbal extract treated and vehicle groups of animals were within normal limits.

Under the conditions of the study, 15 days intra-peritoneal administration of herbal extract in albino mice at the highest dosage level tested containing 200 mg/kg Betulinic acid did not produce any observable untoward toxic effects when compared to untreated group of animals, except for mild inflammation at the site of injection. Hence may be considered as No Observable Effect Level.

N.O.E.L (I.P.)=200 mg/kg

1. A method for treating cancer or a tumor comprising administering an effective amount of betulinic acid rich extract of Zizyphus to a patient in need thereof.

2. The method according to claim 1, wherein the betulinic acid rich extract of Ziziphus is prepared by the process comprising the steps of:
a) optionally drying the bark of Ziziphus;
b) preparing smaller pieces of the bark;
c) pre-macerating the bark in a solvent;
d) recovering the bark and optionally drying the bark;
e) macerating the bark again in a solvent;
f) filtering to recover the solvent; and
g) removing the solvent to obtain the extract.

3. The method according to claim 1, wherein the betulinic acid rich extract of Ziziphus, is prepared by the process comprising the steps of:
a) soaking dried outer bark of Zizyphus in a liquid hydro-alcoholic solution;
b) stirring at room temperature for 12-20 hours to obtain a mixture;
c) filtering or centrifuging to remove the liquid from the mixture, recovering the alcohol from the liquid portion; bark being optionally dried at 100° C. for 6-8 hrs;
d) macerating the bark in alcohol for 12-20 hrs;
e) filtering or centrifuging the macerated mass prepared in step d) to separate the bark material from the liquid;
f) washing the mass obtained in step e) with alcohol;
g) separating the mass from the liquid and discarding the mass;
h) mixing the liquid obtained in step g) with the liquid obtained in step e); and
i) evaporating the mixture prepared in step h) to obtain a dry mass of the betulinic acid rich extract.

4. The method according to claim 1, 2 or 3, wherein the cancer is cancer of the colon, intestine, stomach, breast, melanoma, glioblastoma, lung, cervix, ovary, prostate, oral cavity, larynx, liver, pancreas, kidney, bladder, endothelial cells, leukemia or myeloma.

5. A method for inhibiting Protein Kinase C activity in tumor cells comprising incubating said tumor cells with an amount of extract effective to inhibit Protein Kinase C activity.

6. A method for inhibiting Protein Kinase C activity in tumor cells comprising incubating said tumor cells with an amount of extract of Zizyphus prepared according to the method of claim 2 or 3 effective to inhibit Protein Kinase C activity.

7. A method for inhibiting Protein Kinase C activity in a patient comprising administering to the patient an amount of an extract of Zizyphus effective to inhibit Protein Kinase C activity.

8. A method for inhibiting Protein Kinase C activity in a patient comprising administering to the patient an amount of an extract of Zizyphus effective to inhibit Protein Kinase C activity wherein the extract of Zizyphus is prepared according to the method of claim 2 or 3.

9. A method for inhibiting tumor cell proliferation comprising incubating said cells with an extract of Zizyphus in an amount effective for increasing free nucleosomes and cause apoptosis.

10. A method for inhibiting tumor cell proliferation comprising incubating said cells with an extract of Zizyphus prepared according to the method of claim 2 or 3 in an amount effective for increasing free nucleosomes and cause apoptosis.

11. A method for inhibiting tumor cell proliferation in a patient comprising administering to the patient an amount of an extract of Zizyphus effective to cause apoptosis.

12. A method for inhibiting tumor cell proliferation in a patient comprising administering to the patient an amount of an extract of Zizyphus effective to cause apoptosis wherein the extract of Zizyphus is prepared according to the method of claim 2 or 3.

13. A method for inhibiting tumor cell proliferation by downregulating antiapoptotic protein bcl2 to cause apoptosis comprising incubating said cells with an extract of Zizyphus in an amount effective to downregulate antiapoptotic protein bcl2.

14. A method for inhibiting tumor cell proliferation by downregulating antiapoptotic protein bcl2 to cause apoptosis comprising incubating said cells with an extract of Zizyphus prepared according to the method of claim 2 or 3 in an amount effective to downregulate antiapoptotic protein bcl2.

15. A method for inhibiting tumor cell proliferation by downregulating antiapoptotic protein bcl2 to cause apoptosis comprising administering to a patient an amount of an extract of Zizyphus effective to downregulate antiapoptotic protein bcl2.

16. A method for inhibiting tumor cell proliferation by downregulating antiapoptotic protein bcl2 to cause apoptosis comprising administering to a patient an amount of an extract of Zizyphus effective to downregulate antiapoptotic protein bcl2 wherein the extract of Zizyphus is prepared according to the method of claim 2 or 3.

17. A method for inhibiting tumor cell proliferation by inhibiting vascular endothelial growth factor to cause an antiangiogenic effect comprising incubating said cells with an extract of Zizyphus in an amount effective to inhibit vascular endothelial growth factor.

18. A method for inhibiting tumor cell proliferation by inhibiting vascular endothelial growth factor to cause an antiangiogenic effect comprising incubating said cells with an extract of Zizyphus prepared according to the method of claim 2 or 3 in an amount effective to inhibit vascular endothelial growth factor.

19. A method for inhibiting tumor cell proliferation by inhibiting vascular endothelial growth factor to cause an antiangiogenic effect comprising administering to a patient an amount of an extract of Zizyphus effective to inhibit vascular endothelial growth factor.

20. A method for inhibiting tumor cell proliferation by inhibiting vascular endothelial growth factor to cause an antiangiogenic effect comprising administering to a patient an amount of an extract of Zizyphus effective to inhibit vascular endothelial growth factor wherein the extract of Zizyphus is prepared according to the method of claim 2 or 3.

21. A method for inhibiting tumor cell proliferation by inhibiting basic fibroblast growth factor to cause an antiangiogenic effect comprising incubating said cells with an extract of Zizyphus in an amount effective to inhibit basic fibroblast growth factor.

22. A method for inhibiting tumor cell proliferation by inhibiting basic fibroblast growth factor to cause an antiangiogenic effect comprising incubating said cells with an extract of Zizyphus prepared according to the method of claim 2 or 3 in an amount effective to inhibit basic fibroblast growth factor.

23. A method for inhibiting tumor cell proliferation by inhibiting basic fibroblast growth factor to cause an antiangiogenic effect comprising administering to a patient an amount of an extract of Zizyphus effective to inhibit basic basic fibroblast growth factor.

24. A method for inhibiting tumor cell proliferation by inhibiting basic fibroblast growth factor to cause an antiangiogenic effect comprising administering to a patient an amount of an extract of Zizyphus effective to inhibit basic fibroblast growth factor wherein the extract of Zizyphus is prepared according to the method of claim 2 or 3.

25. A method for inhibiting tumor cell proliferation by upregulating endostatin to cause an antiangiogenic effect comprising incubating said cells with an extract of Zizyphus in an amount effective to upregulate endostatin.

26. A method for inhibiting tumor cell proliferation by upregulating endostatin to cause an antiangiogenic effect comprising incubating said cells with an extract of Zizyphus prepared according to the method of claim 2 or 3 in an amount effective to upregulate endostatin.

27. A method for inhibiting tumor cell proliferation by upregulating endostatin to cause an antiangiogenic effect comprising administering to a patient an amount of an extract of Zizyphus effective to upregulate endostatin.

28. A method for inhibiting tumor cell proliferation by upregulating endostatin to cause an antiangiogenic effect comprising administering to a patient an amount of an extract of Zizyphus effective to upregulate endostatin wherein the extract of Zizyphus is prepared according to the method of claim 2 or 3.

29. The method according to claim 1, 2 or 3, wherein the extract is administered orally to the patient.

30. A method according to claim 1, 2 or 3, wherein the extract of Zizyphus is administered in the form of a tablet, lozenge, capsule, powder, aqueous or oily suspension, emulsion, syrup, elixir, or aqueous solution.

31. A composition of herbal extract of Zizyphus comprising betulinic acid and a pharmaceutically acceptable additive, diluent, excipient, solvent, binder, stabilizer, carrier, filler or lubricant.

32. A composition as claimed in claim 31 which provides 500 mg to 5000 mg per unit dose of betulinic acid in the herbal extract.

33. A method as claimed in claim 1, 2 or 3 wherein said patient is a human, mammal or other animal.

34. A method as claimed in claim 1, 2 or 3 wherein the dosage for humans is in the range of 10 to 200 mg/kg/day.

35. A method as claimed in claim 1, 2 or 3 wherein the extract of Zizyphus is administered to the patient systemically.

36. The method according to claim 1, wherein the treatment comprises administering to the individual a therapeutically effective amount of one or more chemotherapeutic drugs with the extract of Zizyphus.
